(19) United States
(12) Patent Application Publication
(10) Pub. No.: US 2026/0215720 A1
(43) Pub. Date: Jul. 30, 2026

(54) APPARATUS AND METHOD FOR MEDICAL SIGNAL INTERPRETATION

(71) Applicant: Anumana, Inc., Cambridge, MA (US)

(72) Inventors: Uddeshya Upadhyay, Bengaluru (IN); Sairam Bade, Thelangana (IN); Ashim Prasad, Bangalore (IN); Rakesh Barve, Bengaluru (IN)

(73) Assignee: Anumana, Inc., Cambridge, MA (US)

(21) Appl. No.: 19/038,343

(22) Filed: Jan. 27, 2025

(51) Int. Cl. A61B 5/318; A61B 5/256; G06N 20/00

(52) U.S. Cl. CPC A61B 5/318; A61B 5/256; G06N 20/00

(57) ABSTRACT

Disclosed herein is an apparatus and method for medical signal interpretation. An apparatus may train an encoder on a first training dataset comprising a plurality of elements of medical time series training data, generating a predictive machine learning model by combining the encoder with a medical prediction downstream head, and using the predictive machine learning model, generating a medical prediction as a function of medical time series input data.

### FIELD OF THE INVENTION

The present invention generally relates to the field of medical signal interpretation. In particular, the present invention is directed to an apparatus and method for medical signal interpretation.

### BACKGROUND

Electrocardiogram (ECG) data is traditionally analyzed manually by a specialist. ECG data may come in multiple forms, increasing the difficulty of creating a machine learning model capable of interpreting multiple forms of ECG.

### SUMMARY OF THE DISCLOSURE

In an aspect, disclosed herein is an apparatus for medical signal interpretation. Such apparatus may include an electrocardiogram device, wherein the electrocardiogram device is configured to collect medical time series input data from a subject, at least a processor, and a memory communicatively connected to the at least a processor, wherein the memory contains instructions configuring the at least a processor to train an encoder on a first training dataset comprising a plurality of elements of medical time series training data by masking a subset of an element of medical time series training data of the plurality of elements of medical time series training data to generate at least a masked element of medical time series training data and a plurality of unmasked elements of medical time series training data, generating, using an encoder, a plurality of medical time series training data tokens as a function of the plurality of unmasked elements of medical time series training data, generating, using a decoder, a reconstruction of the element of medical time series training data as a function of the plurality of medical time series training data tokens, and modifying the encoder as a function of the element of medical time series training data and the reconstruction of the element of medical time series training data, generating a predictive machine learning model by combining the encoder with a medical prediction downstream head, and using the predictive machine learning model, generating a medical prediction as a function of medical time series input data.

In an aspect, disclosed herein is a method of medical signal interpretation. Such method may include, using at least a processor, training an encoder on a first training dataset comprising a plurality of elements of medical time series training data by masking a subset of an element of medical time series training data of the plurality of elements of medical time series training data to generate at least a masked element of medical time series training data and a plurality of unmasked elements of medical time series training data, generating, using an encoder, a plurality of medical time series training data tokens as a function of the plurality of unmasked elements of medical time series training data, generating, using a decoder, a reconstruction of the element of medical time series training data as a function of the plurality of medical time series training data tokens, and modifying the encoder as a function of the element of medical time series training data and the reconstruction of the element of medical time series training data, using the at least a processor, generating a predictive machine learning model by combining the encoder with a medical prediction downstream head, and using the at least a processor and the predictive machine learning model, generating a medical prediction as a function of medical time series input data.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a diagram depicting an exemplary embodiment of an apparatus for medical signal interpretation;

FIG. 2 is a diagram of an exemplary architecture of an encoder;

FIG. 3 is a diagram of an exemplary process for training an encoder;

FIG. 4 is a block diagram of an exemplary embodiment of a machine-learning model;

FIG. 5 is a schematic diagram of an exemplary embodiment of a neural network;

FIG. 6 is a schematic diagram of an exemplary embodiment of a neural network node;

FIG. 7 is a schematic diagram of training and test-time training for a machine-learning model;

FIG. 8 is a flow diagram depicting an exemplary embodiment of a method of medical signal interpretation; and

FIG. 9 is a block diagram of a computing system that can be used to implement any one or more of the methodologies disclosed herein and any one or more portions thereof. The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for medical signal interpretation. Medical time series training data of a first training dataset may be used to train an encoder. This may be done by training the encoder to produce encodings which, when decoded, provide an accurate reconstruction of medical time series training data. This encoder may be combined with a medical prediction downstream head to generate a predictive machine learning model. This predictive machine learning model may be trained using supervised learning to make accurate medical predictions based on medical time series input data. In some embodiments, parameters of predictive machine learning model may be temporarily modified based on input data.

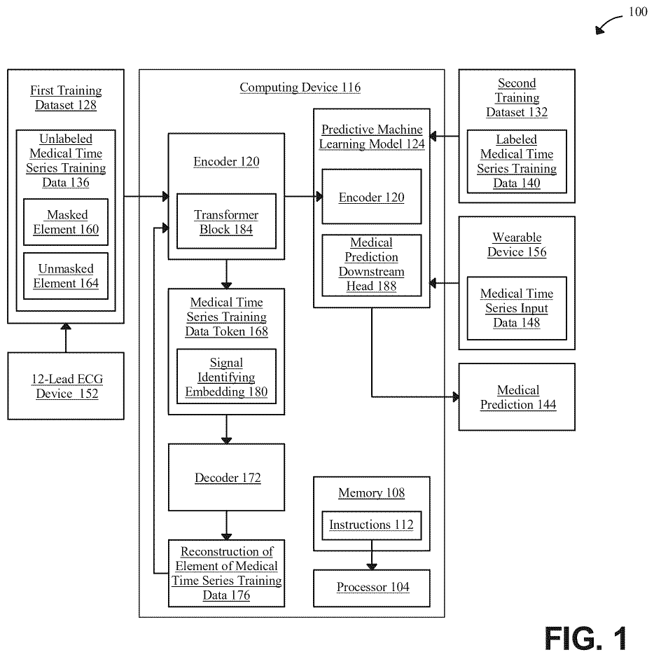

Referring now to FIG. 1, an exemplary embodiment of an apparatus 100 for medical signal interpretation is illustrated. Apparatus 100 may include a computing device. Apparatus 100 may include a processor. Processor may include, without limitation, any processor described in this disclosure. Processor may be included in computing device. Computing device may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device may include a single computing device operating independently, or may include two or more computing device operating in concert, in parallel, sequentially or the like; two or more computing devices may be included together in a single computing device or in two or more computing devices. Computing device may interface or communicate with one or more additional devices as described below in further detail via a network interface device. Network interface device may be utilized for connecting computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

Still referring to FIG. 1, in some embodiments, apparatus 100 may include at least a processor 104 and a memory 108 communicatively connected to the at least a processor 104, the memory 108 containing instructions 112 configuring the at least a processor 104 to perform one or more processes described herein. Computing device 116 may include processor 104 and/or memory 108. Computing device 116 may be configured to perform one or more processes described herein.

Still referring to FIG. 1, computing device 116 may include but is not limited to, for example, a computing device or cluster of computing devices in a first location and a second computing device or cluster of computing devices in a second location. Computing device 116 may include one or more computing devices dedicated to data storage, security, distribution of traffic for load balancing, and the like. Computing device 116 may distribute one or more computing tasks as described below across a plurality of computing devices of computing device, which may operate in parallel, in series, redundantly, or in any other manner used for distribution of tasks or memory between computing devices. Computing device 116 may be implemented, as a non-limiting example, using a “shared nothing” architecture.

Still referring to FIG. 1, computing device 116 may be designed and/or configured to perform any method, method step, or sequence of method steps in any embodiment described in this disclosure, in any order and with any degree of repetition. For instance, computing device 116 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 116 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, as used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, in some embodiments, apparatus 100 may include a user interface. A user interface may be a component of a user device. A user device may include, in non-limiting examples, a smartphone, smartwatch, laptop computer, desktop computer, virtual reality device, or tablet. As used herein, a “user interface” is a mechanism by which a user may input information into a computing device, a mechanism by which a computing device may output information to a user, or both. A user interface may include an input interface and/or an output interface. An input interface may include one or more mechanisms for a computing device to receive data from a user such as, in non-limiting examples, a mouse, keyboard, button, scroll wheel, camera, microphone, switch, lever, touchscreen, trackpad, joystick, and controller. An output interface may include one or more mechanisms for a computing device to output data to a user such as, in non-limiting examples, a screen, speaker, and haptic feedback system. An output interface may be used to display one or more elements of data described herein. As used herein, a device “displays” a datum if the device outputs the datum in a format suitable for communication to a user. For example, a device may display a datum by outputting text or an image on a screen or outputting a sound using a speaker.

Still referring to FIG. 1, apparatus 100 may train encoder 120 and predictive machine learning model 124 using medical time series data, such as medical time series data of first training dataset 128 and/or second training dataset 132. First training dataset 128 may include unlabeled medical time series training data 136. Training of predictive machine learning model 124 using first training dataset 128 may be implemented using a self-supervised learning process. Self-supervised learning processes are described below. In some embodiments, use of self-supervised learning may enable predictive machine learning model 124 to be compatible with different forms of medical time series data, such as different forms of electrocardiogram data. In some embodiments, use of self-supervised learning may allow training without the need for labeled data which may be difficult to generate in sufficient quantities. Second training dataset may include labeled medical time series training data 140. Apparatus 100 may utilize predictive machine learning model 124 to make medical prediction 144 as a function of medical time series input data 148. As used herein, “medical time series data” is a series of values representing medical measurements made at successive times. As used herein, “labeled medical time series training data” is medical time series data which is associated with a label suitable for supervised machine learning. Labeled medical time series training data may include labels such as accurate medical predictions or measurements. Labels may be determined based on, in non-limiting examples, data gathered using a non-ECG medical measurement, such as through use of computed tomography (CT) scan or magnetic resonance imaging (MRI) scan data of a subject which also undergoes an ECG. Use of CT scan and/or MRI scan data for training a machine learning model may be consistent with a process disclosed in U.S. patent application Ser. No. 18/818,311 (having attorney docket number 1518-110USC1), filed on Aug. 28, 2024, and titled “APPARATUS AND METHOD FOR GENERATING A THREE-DIMENSIONAL (3D) MODEL WITH AN OVERLAY,” the entirety of which is hereby incorporated by reference. In another non-limiting example, labels may be determined based on an assessment of ECG data by a medical professional, such as a diagnosis. As used herein, “unlabeled medical time series training data” is medical time series data which is not associated with label suitable for supervised machine learning, medical time series data which is associated with labels suitable for supervised machine learning but for which the labels are not used to train a machine learning model, or both. As used herein, “medical time series input data” is medical time series data which is input into a predictive machine learning model in order to produce a medical prediction. As used herein, a “medical prediction” is an estimation of a medical state of a subject, a prediction of a future medical state of a subject, or both. A medical prediction may include, in non-limiting examples, a determination as to whether a subject has or will in the future have a particular medical condition or category of medical condition, or a likelihood that a subject has or will in the future have a particular medical condition or category of medical condition. A medical condition may be associated with a cardiac health of a subject, and may include, in a non-limiting example, atrial fibrillation (AFib).

Still referring to FIG. 1, in some embodiments, apparatus 100 may include an electrocardiogram (ECG) device. As used herein, an “ECG device” is a device configured to generate ECG data based on an electrical activity of a heart of a subject. In some embodiments, an ECG device may be configured to collect medical time series input data from a subject.

Still referring to FIG. 1, in some embodiments, medical time series data may include electrocardiogram (ECG) data. As used herein, “ECG data” is medical time series data representing electrical activity of a heart. In some embodiments, ECG data may include 12 lead ECG data. In some embodiments, ECG data may be generated using a 12 lead ECG device 152. As used herein, a “12 lead ECG device” is a device configured to generate ECG data from 12 leads. A 12 lead ECG device may be configured to determine a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage based on an electrical activity of a heart of a subject. In some embodiments, ECG data may include a right arm electrode voltage, a left arm electrode voltage, a right leg electrode voltage, a left leg electrode voltage, a V1 electrode voltage, a V2 electrode voltage, a V3 electrode voltage, a V4 electrode voltage, a V5 electrode voltage, and/or a V6 electrode voltage. Such electrode voltages may include voltages measured by individual ECG electrodes. In some embodiments, ECG data may include a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and/or a V6 electrode voltage. A lead I measurement, a lead II measurement, a lead III measurement, a lead aVR measurement, a lead aVL measurement, and/or a lead aVF measurement may include measurements based on voltages measured by multiple ECG electrodes, such as measurements based on a difference between 2 electrodes.

Still referring to FIG. 1, in some embodiments, ECG data may originate from sources other than 12 lead ECGs. For example, an ECG device may include a wearable device 156 such as a smartwatch. As used herein, a “wearable device” is a computing device designed to be worn by a subject on their body. In some embodiments, a wearable device 156 may include one or more electrodes capable of detecting electrical activity of a heart. In some embodiments, a wearable device 156 may be worn on a subject's wrist. In some embodiments, a wearable device 156 may include a single electrode capable of detecting electrical activity of a heart.

Still referring to FIG. 1, in some embodiments, apparatus 100 may train encoder 120 on first training dataset 128, wherein first training dataset 128 comprises a plurality of elements of medical time series data. As used herein, an “encoder” is a component of a machine learning model which is configured to generate a mathematical representation of data which is internal to the model. In some embodiments, an encoder may generate a mathematical representation of medical time series data. As used herein, a “decoder” is a component of a machine learning model which generates an output sequence to a model based on mathematical representations of data which is internal to the model. In some embodiments, a decoder may generate an output sequence based on mathematical representations of medical time series data. Such plurality of elements of medical time series data may include unlabeled medical time series training data 136. In some embodiments, training encoder 120 on first training dataset 128 may include masking a subset of an element of medical time series training data of the plurality of elements of medical time series training data to generate at least a masked element 160 of medical time series training data and a plurality of unmasked elements 164 of medical time series training data. As used herein, data is “masked” when it is selectively hidden from an encoder during a training process. For example, a first subset of data may be masked, a second subset may be unmasked, and the unmasked data may be input into an encoder during a training process. In some embodiments, training encoder 120 on first training dataset 128 may further include generating, using encoder 120, a plurality of medical time series training data tokens 168 as a function of the plurality of unmasked elements 164 of medical time series training data. In some embodiments, training encoder 120 on first training dataset 128 may further include generating, using a decoder 172, a reconstruction of an element of medical time series training data 176 as a function of such plurality of medical time series training data tokens 168. In some embodiments, training encoder 120 on first training dataset 128 may further include modifying encoder 120 as a function of an element of medical time series training data to be reconstructed and this reconstruction of the element of medical time series training data 176.

Still referring to FIG. 1, in some embodiments, training encoder 120 may include use of a masked autoencoder (MAE) training approach which involves reconstruction of an original signal given only a subset of that signal. This may include use of an encoder which maps this subset of the original signal to a latent representation, and a decoder which reconstructs the original signal from the latent representation. In some embodiments, an asymmetric encoder decoder architecture may be used wherein an encoder operates only on a subset of the original signal, without mask tokens, and a decoder may be used to reconstruct the full signal based on the latent representation and mask tokens.

Still referring to FIG. 1, in some embodiments, masking medical time series training data of first training dataset 128 may include dividing an element of medical time series training data into a first subset, including components to be masked and a second subset, including components not to be masked. In some embodiments, medical time series training data of first training dataset 128 may include 12 lead ECG data, and components to be masked or not to be masked may be selected from a right arm electrode voltage, a left arm electrode voltage, a right leg electrode voltage, a left leg electrode voltage, a V1 electrode voltage, a V2 electrode voltage, a V3 electrode voltage, a V4 electrode voltage, a V5 electrode voltage, and a V6 electrode voltage. In some embodiments, medical time series training data of first training dataset 128 may include 12 lead ECG data, and components to be masked or not to be masked may be selected from a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage. In some embodiments, which components are masked, and which are unmasked are selected randomly. In some embodiments, which components are masked, and which are unmasked are selected independently for each training example. In some embodiments, 1, 2, 3, 4, 5, 6, 7, 8, 9, 10, or 11 leads of 12 lead ECG data are masked. In some embodiments, 1, 2, 3, 4, 5, 6, 7, 8, or 9 electrodes of 12 lead ECG data are masked.

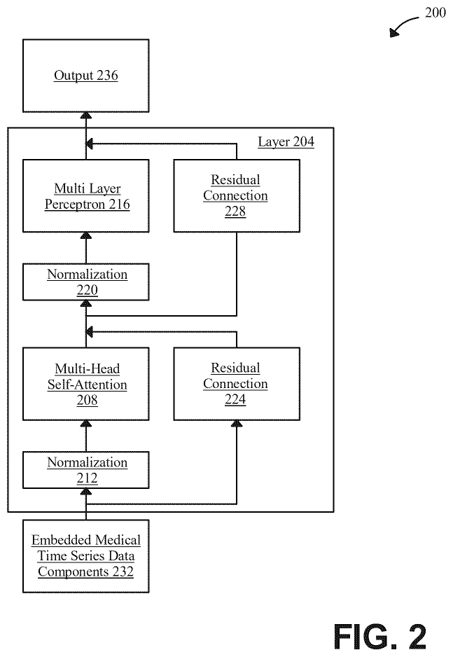

Still referring to FIG. 1, in some embodiments, encoder 120 is only applied to unmasked components of medical time series training data of first dataset 128. In some embodiments, masking tokens are not input into encoder 120. In some embodiments, not inputting masking tokens into encoder 120 may reduce computational and/or memory needs of training encoder 120. This may allow for faster computing. In some embodiments, encoder 120 may embed data using a linear projection with added signal identifying embeddings 180. As used herein, a “signal identifying embedding” is a component of a medical time series training data token which identifies which of a set of components of medical time series training data is represented by a medical time series training data token, a mask token or a component thereof. In a non-limiting example, medical time series training data may include 12 lead ECG data including a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage, and a signal identifying embedding 180 may identify that medical time series training data token 168 or a component thereof represents a lead II measurement. This information may influence a reconstruction of element of medical time series training data 176 by decoder 172. As described below, medical time series training data token 168 may be generated by processing one or more medical time series training data pre-token data using one or more transformer blocks 184. As used herein, a “medical time series training data pre-token datum” is a datum internal to an encoder which is generated based on an element of medical time series training data, and which is used to generate a medical time series training data token. As used herein, a “transformer block” is a neural network architecture which uses self-attention and positional encoding. Such positional encoding may include a signal identifying embedding. In some embodiments, a medical time series training data pre-token datum may be generated by a linear projection and adding a signal identifying embedding 180. Medical time series training data pre-token datum may then be processed using a transformer block. In some embodiments, multiple transformer blocks may be used. Application of transformer blocks is described with respect to FIG. 2.

Still referring to FIG. 1, in some embodiments, decoder 172 may receive, as inputs, encoded components of medical time series data, and mask tokens. Mask tokens may include shared, learned vectors that each indicate the presence of a missing component (such as a missing ECG lead) to be predicted. Signal identifying embeddings 180 may be added to such data. For example, signal identifying embeddings may be added to encoded components of medical time series data and/or mask tokens. In some embodiments, decoder 172 may include one or more transformer blocks as described below. In some embodiments, decoder 172 is used only in the training of encoder 120 and is not used in generating medical prediction 144 from medical time series input data 148.

Still referring to FIG. 1, in some embodiments, decoder 172 may reconstruct an element of medical time series training data by predicting values for one or more components of medical time series training data at particular times. For example, decoder 172 may predict a value for a V3 electrode voltage at a particular time. In some embodiments, decoder 172 may be limited to reconstructing medical time series training data of a particular duration. In some embodiments, a last layer of decoder 172 may include a linear projection whose number of output channels equals the number of measurements to be predicted. In a non-limiting example, this may include a number of output channels equal to a number of points in time at which a measurement is to be predicted times a number of components whose measurements are to be predicted. In some embodiments, decoder 172's output is reshaped to form a reconstruction of element of medical time series training data 176. In some embodiments, a loss function used to modify encoder 120 may compute a mean squared error between reconstruction of element of medical time series training data 176 and a corresponding original element of medical time series training data. In some embodiments, loss is only computed on reconstruction of masked components. In some embodiments, a reconstruction target may include normalized pixel values of a masked component. In a non-limiting example, a mean and standard deviation of all measurements of a component may be computed and used to normalize a component.

Still referring to FIG. 1, in some embodiments, encoder 120 may include transformer block 184. In some embodiments, generation of a plurality of medical time series training data tokens 168 may include processing a medical time series training data token using a transformer block 184. In some embodiments, transformer block 184 may accept as an input medical time series training data which is split into components and linearly embedded. Encoder 120 may include a plurality of transformer blocks, with each block including a multi-head self-attention block and a multi-layer perceptron (MLP) block. In some embodiments, multi-head self-attention block and/or MLP block may include layer normalization. In some embodiments, in the context of medical time series training data including ECG data, transformer block 184 may be used to add context to medical time series data tokens 168 based on other tokens comprising different electrodes or different leads.

Still referring to FIG. 1, in some embodiments, apparatus 100 may combine encoder 120 with medical prediction downstream head 188 in order to generate predictive machine learning model 124. As used herein, a “predictive machine learning model” is a machine learning model trained to produce a medical prediction. As used herein, a “medical prediction downstream head” is a component of a predictive machine learning model, where an input of the component is generated using an encoder, and where the component produces as an output a medical prediction.

Still referring to FIG. 1, in some embodiments, predictive machine learning model 124 and/or medical prediction downstream head 188 may be trained using supervised learning. Predictive machine learning model 124 and/or medical prediction downstream head 188 may be trained using a supervised learning algorithm. Predictive machine learning model 124 and/or medical prediction downstream head 188 may include a classifier. For example, predictive machine learning model 124 may classify medical time series input data 148 to particular medical conditions. Predictive machine learning model 124 and/or medical prediction downstream head 188 may include a neural network. Predictive machine learning model 124 and/or medical prediction downstream head 188 may be trained on second training dataset 132 including labeled medical time series training data 140. labeled medical time series training data 140 may include example medical time series data, associated with example medical predictions. Such medical predictions may include, in non-limiting examples, a determination as to whether a subject has or will in the future have a particular medical condition or category of medical condition, or a likelihood that a subject has or will in the future have a particular medical condition or category of medical condition. Once predictive machine learning model 124 and/or medical prediction downstream head 188 is trained, it may be used to determine medical prediction 144. Apparatus 100 may input medical time series input data 148 into predictive machine learning model 124, and apparatus 100 may receive medical prediction 144 from the model.

Still referring to FIG. 1, in some embodiments, encoder 120 and medical prediction downstream head 188 may be trained via fine-tuning. For example, encoder 120 and medical prediction downstream head 188 may be trained together end to end to make medical prediction 144. In some embodiments, encoder 120 may be used as a fixed feature extractor, and only medical prediction downstream head 188 is trained, such as by linear probing.

Still referring to FIG. 1, in some embodiments, predictive machine learning model 124 may be trained using test time training. In some embodiments, generation of medical prediction 144 may include modifying a parameter of predictive machine learning model 124 as a function of medical time series input data 148. In some embodiments, modifying a parameter of predictive machine learning model 124 may include finetuning encoder 120 as a function of a sample comprising a plurality of elements of medical time series input data 148 and generating medical prediction 144 using a resulting finetuned encoder.

Still referring to FIG. 1, in some embodiments, predictive machine learning model 124 may be trained using a Y shaped architecture as follows: encoder f may be followed on one branch by decoder g and on a second branch by main task head h. Main task head h may use a linear projection from a dimension of encoder f created features to a number of classes. Encoder f may be pre-trained as described herein, such as through training an encoder and decoder to reconstruct medical time series training data.

Still referring to FIG. 1, in some embodiments, prior to test time, predictive machine learning model 124 may be trained by fine tuning. For example, for main task head h and encoder f, h∘f may be trained end to end.

Still referring to FIG. 1, in some embodiments, prior to test time, for main task head h, encoder f, and decoder g, both h∘f and g∘f may be trained by summing their losses together.

Still referring to FIG. 1, in some embodiments, prior to test time, main task head h may be trained, and encoder f may be kept frozen. This may be implemented by producing a trained main task head h₀ using the equation

$${h}_{0} = arg {min}_{h} \frac{1}{n} {\sum }_{i = 1}^{n} {l}_{m} ( h ∘ {f}_{0} ( {x}_{i} ) , {y}_{i} ) .$$

This summation is over a training set with n samples, each including input xᵢ and label yᵢ. Main task loss lₘ may in some embodiments include cross entropy loss. This method may be used to train main task head h₀ while encoder f₀ may be frozen.

Still referring to FIG. 1, in some embodiments, test time training may include optimizing the following loss: fₓ, gₓ=arg min_{f,g}lₛ(g∘f(mask(x)), x), where h₀ is a main task head, f₀ is a pre trained encoder, and g₀ is a decoder. Self-supervised reconstruction loss is may compute error of decoded components of medical time series data relative to ground truth. After such test time training, a prediction may be made on x as h∘fₓ(x). In some embodiments, modifying a parameter of predictive machine learning model may include performing a gradient based optimization based on an error of decoded components of medical time series data relative to ground truth. In some embodiments, gradient based optimization at test time training may start with f₀ and g₀, and fₓ and gₓ may be discarded after a prediction is made on each test input x, and weights may be reset to f₀ and g₀ for the next test input. In some embodiments, a modified parameter of the predictive machine learning model may be reset to a pre-test time training state after the medical prediction is generated.

Still referring to FIG. 1, in some embodiments, predictive machine learning model 124 and/or encoder 120 may be trained using an objective function based on both self-supervised auxiliary task loss and main task loss. Self-supervised auxiliary task loss may include loss generated based on the model's recognition of a mathematical transformation applied to input data. For example, self-supervised auxiliary task loss may include loss generated based on the degree to which the model accurately determines which of a plurality of possible mathematical transformations is applied to input data. Main task loss may include loss generated based on supervised learning, such as supervised learning. Supervised learning may be implemented as described herein with respect to second training dataset 132 and labeled medical time series training data 140. In some embodiments, predictive machine learning model 124 and/or encoder 120 may be trained using an objective function in which losses for self-supervised auxiliary task loss and main task loss are added together and gradients are determined based on such combined losses.

Still referring to FIG. 1, in some embodiments, apparatus 100 may train predictive machine learning model 124 as described herein, and once predictive machine learning model 124 is trained, may use predictive machine learning model 124 to generate medical prediction 144 as a function of medical time series input data 148. As used herein, “test time training” is a process including (1) modification of one or more parameters of a machine learning model based on an input to the model, and (2) use of a resulting modified machine learning model to generate an output based on the input. In some embodiments, training predictive machine learning model 124 using test time training may improve the accuracy of a model output when the test time input data comes from a different distribution than a training dataset.

Still referring to FIG. 1, in some embodiments, training data such as first training dataset 128 and/or second training dataset 132 may be split into one or more subsets. In some embodiments, a training data subset may include a training set, which may be used to directly train model parameters. In some embodiments, a training data subset may include a validation set, which may be used to evaluate a model trained on a training set. In some embodiments, one or more aspects of a model may be determined based on performance of a model's performance on a validation set. In non-limiting examples, a learning rate may be changed, a learning algorithm may be changed, or a feature may be added or removed based on model performance on a validation set. In some embodiments, a training data subset may include a test set. In some embodiments, a test set may be used to evaluate a model which performs sufficiently well on a validation set. In some embodiments, splitting of training data into subsets may be performed such that each subset is representative of the entire data set. In a non-limiting example, splitting of training data into subsets may be performed such that which instances of data enter which subset is determined randomly. In some embodiments, data may be split into subsets at a predetermined ratio. In a non-limiting example, training data may be split such that 70% of the training data is in a training set, 15% is in a validation set, and 15% is in a test set. In some embodiments, a model is not trained on data in a test set and/or a validation set. In some embodiments, there is no overlap between data in a training set, a test set, and a validation set. In some embodiments, splitting training data into multiple subsets may reduce overfitting.

Referring now to FIG. 2, a diagram of an exemplary architecture of encoder 200 is provided. Encoder 200 may include one or more layers 204. In some embodiments, layers may be identical in structure. In some embodiments, a layer 204 may include a first sub-layer including multi-head self-attention 208 and a normalization function 212, and a second sub-layer including a multi-layer perceptron (MPL) 216 block and a normalization function 220. In some embodiments, a residual connection, such as residual connection 224 and/or residual connection 228 around one or more sub-layers may be used such that the output of a sub-layer is LayerNorm(x+Sublayer(x)), where Sublayer(x) is the function implemented by the sub-layer, and x is an input. In some embodiments, the dimensions of input and output layers of encoder sub-layers may be consistent.

Still referring to FIG. 2, in some embodiments, embedded medical time series data components 232 may include a mapping of medical time series data components to D dimensions using a linear projection. In some embodiments, D is a constant latent vector size used through all layers of a transformer.

Still referring to FIG. 2, in some embodiments, self-attention (SA) and/or multihead self-attention (MSA) may be implemented as follows. Self-attention may include qkv self-attention. For each element in input sequence z∈ [symbol] ^{n×D}, where N is a number of components of medical time series data (such as a number of leads, where medical time series data includes ECG data), compute a weighted sum over all values v in the sequence. Attention weights Aᵢⱼ are based on the pairwise similarity between two elements of the sequence and their respective query qⁱ and key k^{j} representations.

$$\begin{matrix}[ q , k , v ] = z {U}_{q k v} \\ A = softmax ( q {k}^{T} / \sqrt{{D}_{h}} ) \\ SA ( z ) = A v\end{matrix} \qquad \begin{matrix}{U}_{q k v} ∈ {ℝ}^{D \times 3 {D}_{h}} \\ A ∈ {ℝ}^{N \times N} \\ \end{matrix}$$

Still referring to FIG. 2, multihead self-attention may include an extension of self-attention in which k self-attention operations, called “heads”, are operated in parallel, and their concatenated outputs are projected. In some embodiments, Dₕ may be set to D/k.

$$MSA ( z ) = [ S {A}_{1} ( z ) ; S {A}_{2} ( z ) ; … ; {SA}_{k} ( z ) ] {U}_{m s a} \qquad {U}_{m s a} ∈ {ℝ}^{k \cdot {D}_{h} \times D}$$

Still referring to FIG. 2, in some embodiments, a multi-layer perceptron sub-layer may include one or more (in some embodiments, two) layers with a GELU non-linearity.

Still referring to FIG. 2, in some embodiments, output 236 may be generated based on the outputs of multi-layer perceptron 216 and/or residual connection 228. In some embodiments, output 236 may be used as an input to another transformer layer. In some embodiments, output 236 may include an embedding which may be decoded using a decoder as described above.

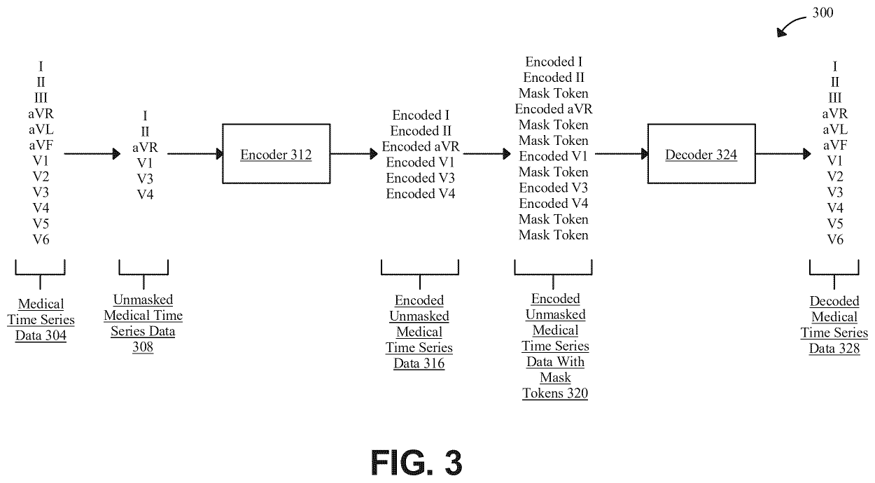

Referring now to FIG. 3, a diagram of an exemplary process 300 for training an encoder is provided. Medical time series data 304 may include, for example, components of ECG data, such as readings of ECG leads. A subset of such leads may be masked, such that they are not input into encoder 312. The remaining unmasked medical time series data 308 may be input into encoder 312, which may output encoded unmasked medical time series data 316. Encoded unmasked medical time series data 316 may include encodings corresponding to components of medical time series data of unmasked medical time series data 308. Mask tokens may be added to encoded unmasked medical time series data 316 to generate encoded unmasked time series data with mask tokens 320. Encoded unmasked time series data with mask tokens 320 may include signal identifying embeddings such that, for example, which leads are represented by which embeddings and/or mask tokens. Encoded unmasked time series data with mask tokens 320 may be input into decoder 324, which may output decoded medical time series data 328. Encoder 312 may be trained to improve accuracy of decoded medical time series data 328 with respect to medical time series data 304. In some embodiments, data input into encoder 312 may include input data consisting of a plurality of unmasked elements of medical time series training data.

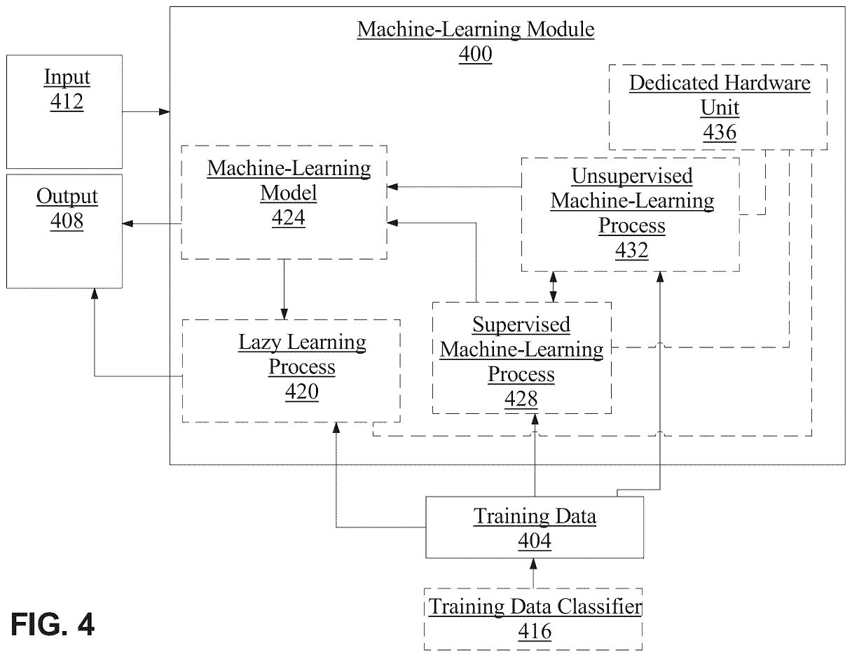

Referring now to FIG. 4, an exemplary embodiment of a machine-learning module 400 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 404 to generate an algorithm instantiated in hardware or software logic, data structures, and/or functions that will be performed by a computing device/module to produce outputs 408 given data provided as inputs 412; this is in contrast to a non-machine learning software program where the commands to be executed are determined in advance by a user and written in a programming language.

Still referring to FIG. 4, “training data,” as used herein, is data containing correlations that a machine-learning process may use to model relationships between two or more categories of data elements. For instance, and without limitation, training data 404 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 404 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 404 according to various correlations; correlations may indicate causative and/or predictive links between categories of data elements, which may be modeled as relationships such as mathematical relationships by machine-learning processes as described in further detail below. Training data 404 may be formatted and/or organized by categories of data elements, for instance by associating data elements with one or more descriptors corresponding to categories of data elements. As a non-limiting example, training data 404 may include data entered in standardized forms by persons or processes, such that entry of a given data element in a given field in a form may be mapped to one or more descriptors of categories. Elements in training data 404 may be linked to descriptors of categories by tags, tokens, or other data elements; for instance, and without limitation, training data 404 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 4, training data 404 may include one or more elements that are not categorized; that is, training data 404 may not be formatted or contain descriptors for some elements of data. Machine-learning algorithms and/or other processes may sort training data 404 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 404 to be made applicable for two or more distinct machine-learning algorithms as described in further detail below. Training data 404 used by machine-learning module 400 may correlate any input data as described in this disclosure to any output data as described in this disclosure. As a non-limiting illustrative example, an input may include medical time series input data and an output may include a medical prediction.

Further referring to FIG. 4, training data may be filtered, sorted, and/or selected using one or more supervised and/or unsupervised machine-learning processes and/or models as described in further detail below; such models may include without limitation a training data classifier 416. Training data classifier 416 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 400 may generate a classifier using a classification algorithm, defined as a processes whereby a computing device and/or any module and/or component operating thereon derives a classifier from training data 404. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 416 may classify elements of training data to, for example, a particular category of ECG input data.

Still referring to FIG. 4, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 4, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 4, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 4, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 4, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 4, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 4, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 4, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 4, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 4, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 4, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{n e w} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{n e w} = \frac{X - {X}_{m e a n}}{{X}_{max} - {X}_{min}} .$$

Feature scaling may include standardization, where a difference between X and Xₘₑₐₙ is divided by a standard deviation σ of a set or subset of values:

$${X}_{n e w} = \frac{X - {X}_{m e a n}}{σ} .$$

Scaling may be performed using a median value of a a set or subset X_{median} and/or interquartile range (IQR), which represents the difference between the 25^{th} percentile value and the 50^{th} percentile value (or closest values thereto by a rounding protocol), such as:

$${X}_{n e w} = \frac{X - {X}_{m e d i a n}}{IQR} .$$

Persons skilled in the art, upon reviewing the entirety of this disclosure, will be aware of various alternative or additional approaches that may be used for feature scaling.

Further referring to FIG. 4, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 4, machine-learning module 400 may be configured to perform a lazy-learning process 420 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 404. Heuristic may include selecting some number of highest-ranking associations and/or training data 404 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 4, machine-learning processes as described in this disclosure may be used to generate machine-learning models 424. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 424 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 424 may be generated by creating an artificial neural network, such as a convolutional neural network comprising an input layer of nodes, one or more intermediate layers, and an output layer of nodes. Connections between nodes may be created via the process of “training” the network, in which elements from a training data 404 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 4, machine-learning algorithms may include at least a supervised machine-learning process 428. At least a supervised machine-learning process 428, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include medical time series input data as described above as inputs, medical predictions as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 404. Persons skilled in the art, upon reviewing the entirety of this disclosure, will be aware of various possible variations of at least a supervised machine-learning process 428 that may be used to determine relation between inputs and outputs. Supervised machine-learning processes may include classification algorithms as defined above.

With further reference to FIG. 4, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 4, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 4, machine learning processes may include at least an unsupervised machine-learning processes 432. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 432 may not require a response variable; unsupervised processes 432 may be used to find interesting patterns and/or inferences between variables, to determine a degree of correlation between two or more variables, or the like.

Still referring to FIG. 4, machine-learning module 400 may be designed and configured to create a machine-learning model 424 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 4, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 4, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 4, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 4, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 4, one or more processes or algorithms described above may be performed by at least a dedicated hardware unit 436. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 436 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 436 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 436 to perform one or more operations described herein, such as evaluation of model and/or algorithm outputs, one-time or iterative updates to parameters, coefficients, weights, and/or biases, and/or any other operations such as vector and/or matrix operations as described in this disclosure.

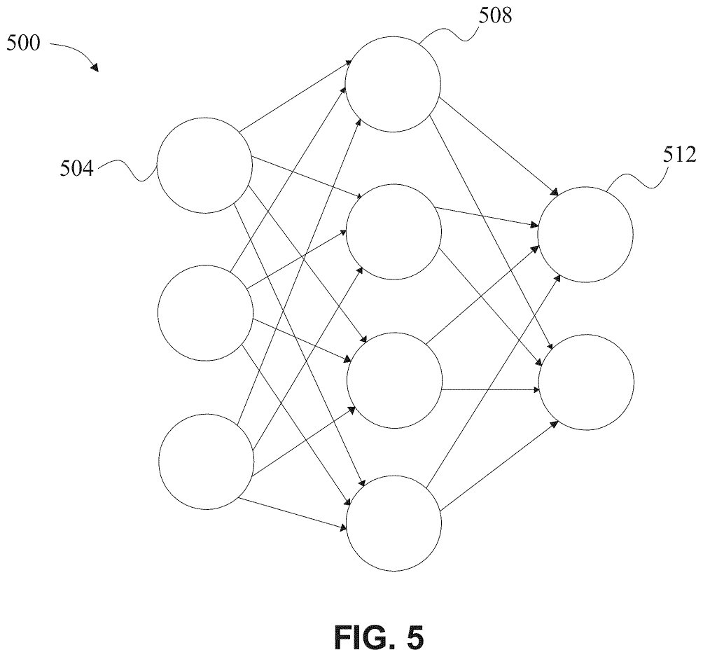

Referring now to FIG. 5, an exemplary embodiment of neural network 500 is illustrated. A neural network 500 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 504, one or more intermediate layers 508, and an output layer of nodes 512. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes.

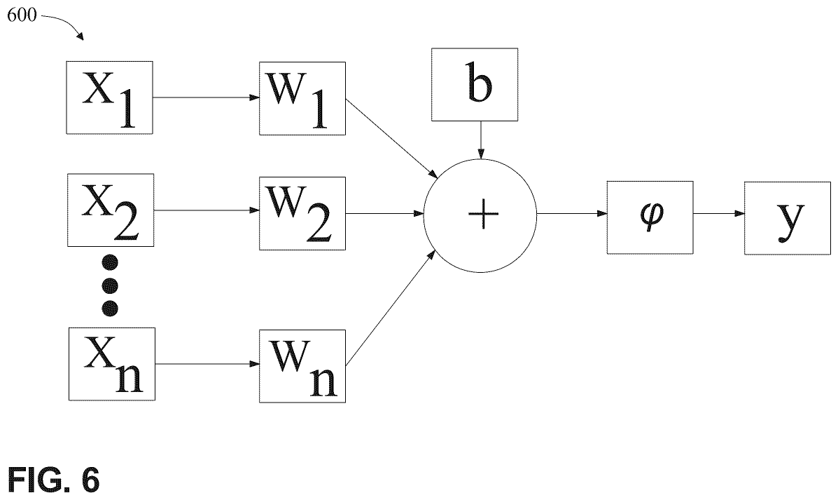

Referring now to FIG. 6, an exemplary embodiment of a node 600 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

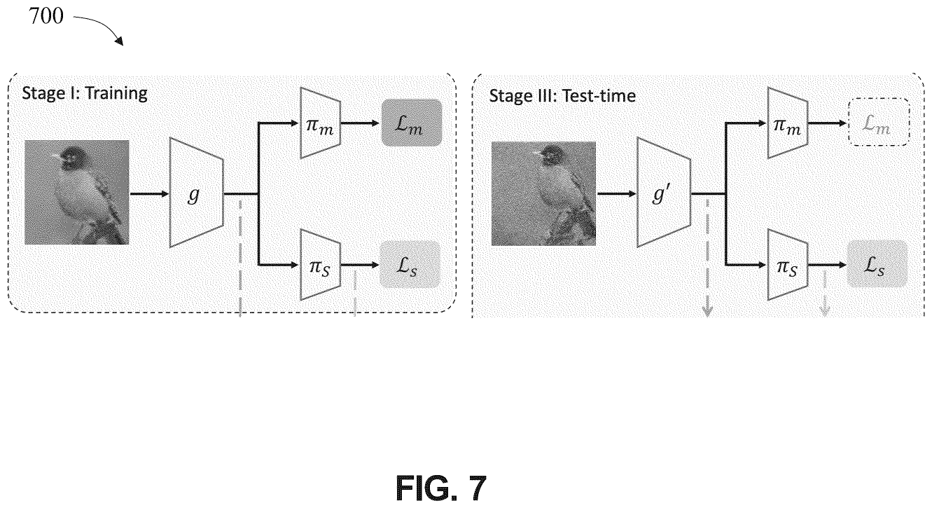

Referring now to FIG. 7, an exemplary schematic 700 of training and test-time training for a machine—m learning model is shown. During a first stage of training, both the main task (πₘ) and auxiliary SSL task (πₛ) are trained. This may include a traditional training step for a machine-learning model, wherein the model is trained on a training dataset. During a subsequent stage of training (depicted as “Stage III” in FIG. 7), machine-learning model may undergo test-time training. Test-time training is a strategy used in machine learning to adapt a model to the specific characteristics of the test data during inference. This approach may improve machine-learning model's performance by leveraging additional information available at test time. During test-time training, the machine-learning model may undergo additional training using the test dataset (or a portion thereof). Implementing test-time training may allow further refinement of a model to suit particular properties of the test data set. In some embodiments, a machine-learning model may include an auxiliary task and a main task. A “main task,” for the purposes of this disclosure, of a machine-learning model, is a task that represents the primary objective of the machine-learning model. An “auxiliary task,” for the purposes of this disclosure, of a machine-learning model, is a task that is learned alongside the main task of the machine-learning model, designed to improve the machine-learning model's performance on the main task by helping it develop better representations from the data. During test-time training, the machine-learning model may update itself based on the auxiliary task using the test data. In some embodiments, during test-time training, an auxiliary SSL task (πₛ) may be back propagated for few steps before finally applying the machine-learning model to get results for the main task (πₘ).

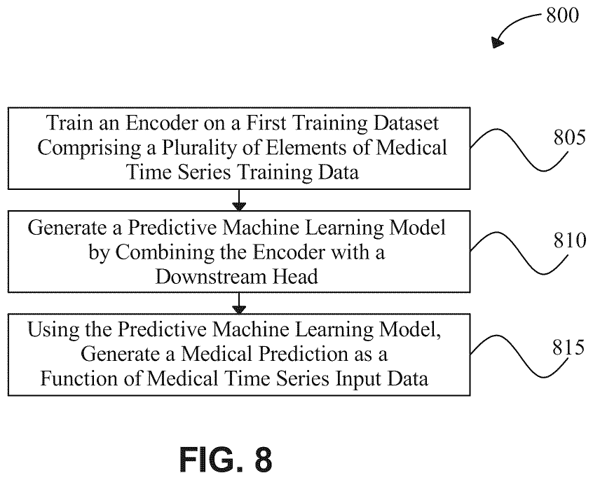

Referring now to FIG. 8, an exemplary embodiment of a method 800 of medical signal interpretation is illustrated. One or more steps if method 800 may be implemented, without limitation, as described with reference to other figures. One or more steps of method 800 may be implemented, without limitation, using at least a processor.

Still referring to FIG. 8, in some embodiments, method 800 may include a step 805 of training an encoder on a first training dataset comprising a plurality of elements of medical time series training data. In some embodiments, this step may include masking a subset of an element of medical time series training data of the plurality of elements of medical time series training data to generate at least a masked element of medical time series training data and a plurality of unmasked elements of medical time series training data. In some embodiments, this step may include generating, using an encoder, a plurality of medical time series training data tokens as a function of the plurality of unmasked elements of medical time series training data. In some embodiments, this step may include generating, using a decoder, a reconstruction of the element of medical time series training data as a function of the plurality of medical time series training data tokens. In some embodiments, this step may include modifying the encoder as a function of the element of medical time series training data and the reconstruction of the element of medical time series training data. In some embodiments, generation of the plurality of medical time series training data tokens comprises processing a medical time series training data pre-token datum using a transformer block. In some embodiments, the plurality of elements of medical time series training data comprises 12-lead electrocardiogram (ECG) data. In some embodiments, the ECG data comprises a right arm electrode voltage, a left arm electrode voltage, a right leg electrode voltage, a left leg electrode voltage, a V1 electrode voltage, a V2 electrode voltage, a V3 electrode voltage, a V4 electrode voltage, a V5 electrode voltage, and a V6 electrode voltage. In some embodiments, the ECG data comprises a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage. In some embodiments, masking a subset of an element of medical time series training data comprises masking a lead of the 12-lead electrocardiogram data. In some embodiments, generating, using a decoder, a reconstruction of the element of medical time series training data comprises inputting into the decoder a mask token. In some embodiments, training an encoder on a first training dataset may include inputting into the encoder input data consisting of the plurality of unmasked elements of medical time series training data.

Still referring to FIG. 8, in some embodiments, method 800 may include a step 810 of generating a predictive machine learning model by combining the encoder with a medical prediction downstream head.

Still referring to FIG. 8, in some embodiments, method 800 may include a step 815 of using the predictive machine learning model, generating a medical prediction as a function of medical time series input data. In some embodiments, generating the medical prediction comprises modifying a parameter of the predictive machine learning model as a function of the medical time series input data. In some embodiments, modifying a parameter of the predictive machine learning model comprises performing a gradient based optimization based on an error of decoded components of medical time series data relative to ground truth. In some embodiments, the method further comprises resetting a modified parameter of the predictive machine learning model to a pre-test time training state after the medical prediction is generated. In some embodiments, modifying a parameter of the predictive machine learning model comprises finetuning the encoder as a function of a sample comprising a plurality of elements of the medical time series input data and generating the medical prediction using a resulting finetuned encoder. In some embodiments, the medical time series input data comprises ECG data derived from a wearable device 156.

Still referring to FIG. 8, in some embodiments, generating the predictive machine learning model further comprises training the predictive machine learning model on a second training dataset comprising labeled medical time series training data. In some embodiments, the second training dataset comprises ECG data derived from a wearable device 156.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

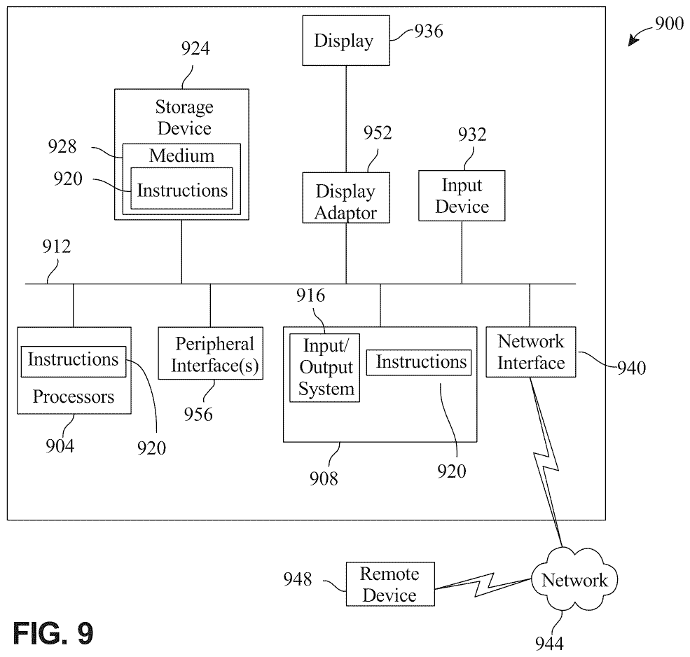

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display 936. Examples of a display device include, but are not limited to, a liquid crystal display (LCD), a cathode ray tube (CRT), a plasma display, a light emitting diode (LED) display, and any combinations thereof. Display adapter 952 and display 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for medical signal interpretation, the apparatus comprising:
an electrocardiogram device comprising a 12 lead electrocardiogram device, wherein the electrocardiogram device is configured to collect medical time series input data from a subject comprising at least a right arm electrode voltage, a left arm electrode voltage and a V1 electrode voltage;
at least a processor; and
a memory communicatively connected to the at least a processor, wherein the memory contains instructions configuring the at least a processor to:
train an encoder on a first training dataset comprising a plurality of elements of medical time series training data by:
masking a subset of an element of medical time series training data of the plurality of elements of medical time series training data to generate at least a masked element of medical time series training data, wherein the medical time series training data of the first training dataset comprises 12 lead ECG data and components selected from the at least a right arm electrode voltage, left arm electrode voltage and a V1 electrode voltage, and a plurality of unmasked elements of medical time series training data;
generating, using an encoder, a plurality of medical time series training data tokens as a function of the plurality of unmasked elements of medical time series training data wherein the encoder is applied to the unmasked elements of medical time series training data to reduce computation needs of training the encoder;
generating, using a decoder, a reconstruction of the element of medical time series training data as a function of the plurality of medical time series training data tokens; and
modifying the encoder as a function of the element of medical time series training data and the reconstruction of the element of medical time series training data, wherein the encoder is modified by the at least a processor operating on 12-lead electrocardiogram signals acquired from the subject;
generating a predictive machine learning model by combining the encoder with a medical prediction downstream head; and
using the predictive machine learning model, generating a medical prediction as a function of the medical time series input data.

2. The apparatus of claim 1, wherein generation of the plurality of medical time series training data tokens comprises processing a medical time series training data pre-token datum using a transformer block.

3. The apparatus of claim 1, wherein generating the medical prediction comprises modifying a parameter of the predictive machine learning model as a function of the medical time series input data.

4. The apparatus of claim 3, wherein modifying a parameter of the predictive machine learning model comprises finetuning the encoder as a function of a sample comprising a plurality of elements of the medical time series input data and generating the medical prediction using a resulting finetuned encoder.

5. The apparatus of claim 3, wherein:
modifying a parameter of the predictive machine learning model comprises performing a gradient based optimization based on an error of decoded components of medical time series data relative to ground truth; and
the memory contains instructions configuring the at least a processor to reset a modified parameter of the predictive machine learning model to a pre-test time training state after the medical prediction is generated.

6. The apparatus of claim 1, wherein the plurality of elements of medical time series training data comprises 12-lead electrocardiogram (ECG) data.

7. The apparatus of claim 6, wherein the ECG data comprises a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage.

8. The apparatus of claim 6, wherein masking a subset of an element of medical time series training data comprises masking a lead of the 12-lead electrocardiogram data.

9. The apparatus of claim 1, wherein generating the predictive machine learning model further comprises training the predictive machine learning model on a second training dataset comprising labeled medical time series training data.

10. The apparatus of claim 9, wherein the second training dataset comprises ECG data derived from a wearable device.

11. The apparatus of claim 1, wherein generating, using a decoder, a reconstruction of the element of medical time series training data comprises inputting into the decoder a mask token.

12. The apparatus of claim 1, wherein training an encoder on a first training dataset comprises inputting into the encoder input data consisting of the plurality of unmasked elements of medical time series training data.

13. A method of medical signal interpretation, the method comprising:
an electrocardiogram device comprising a 12 lead electrocardiogram device, wherein the electrocardiogram device is configured to collect medical time series input data from a subject comprising at least a right arm electrode voltage, a left arm electrode voltage and a V1 electrode voltage;
using at least a processor, training an encoder on a first training dataset comprising a plurality of elements of medical time series training data by:
masking a subset of an element of medical time series training data of the plurality of elements of medical time series training data to generate at least a masked element of medical time series training data wherein the medical time series training data of the first training dataset comprises 12 lead ECG data and components selected from the at least a right arm electrode voltage, left arm electric voltage and a V1 electrode voltage and a plurality of unmasked elements of medical time series training data;
generating, using an encoder, a plurality of medical time series training data tokens as a function of the plurality of unmasked elements of medical time series training data wherein the encoder is applied to the unmasked elements of medical time series training data to reduce computation needs of training the encoder;
generating, using a decoder, a reconstruction of the element of medical time series training data as a function of the plurality of medical time series training data tokens; and
modifying the encoder as a function of the element of medical time series training data and the reconstruction of the element of medical time series training data, wherein the encoder is modified by the at least a processor operating on 12-lead electrocardiogram signals acquired from the subject;
using the at least a processor to generate a predictive machine learning model by combining the encoder with a medical prediction downstream head; and
using the at least a processor and the predictive machine learning model to generate a medical prediction as a function of medical time series input data.

14. The method of claim 13, wherein generation of the plurality of medical time series training data tokens comprises processing a medical time series training data pre-token datum using a transformer block.

15. The method of claim 13, wherein generating the medical prediction comprises modifying a parameter of the predictive machine learning model as a function of the medical time series input data.

16. The method of claim 15, wherein modifying a parameter of the predictive machine learning model comprises finetuning the encoder as a function of a sample comprising a plurality of elements of the medical time series input data and generating the medical prediction using a resulting finetuned encoder.

17. The method of claim 15, wherein:
modifying a parameter of the predictive machine learning model comprises performing a gradient based optimization based on an error of decoded components of medical time series data relative to ground truth; and
the method further comprises resetting a modified parameter of the predictive machine learning model to a pre-test time training state after the medical prediction is generated.

18. The method of claim 13, wherein the plurality of elements of medical time series training data comprises 12-lead electrocardiogram (ECG) data.

19. The method of claim 18, wherein the ECG data comprises a lead I measurement, a lead II measurement, a lead III measurement, a lead aVR measurement, a lead aVL measurement, a lead aVF measurement, a V1 electrode voltage, a V2 electrode voltage, a V3 electrode voltage, a V4 electrode voltage, a V5 electrode voltage, and a V6 electrode voltage.

20. The method of claim 18, wherein masking a subset of an element of medical time series training data comprises masking a lead of the 12-lead electrocardiogram data.

21. The method of claim 13, wherein generating the predictive machine learning model further comprises training the predictive machine learning model on a second training dataset comprising labeled medical time series training data.

22. The method of claim 21, wherein the second training dataset comprises ECG data derived from a wearable device.

23. The method of claim 13, wherein generating, using a decoder, a reconstruction of the element of medical time series training data comprises inputting into the decoder a mask token.

24. The method of claim 13, wherein training an encoder on a first training dataset comprises inputting into the encoder input data consisting of the plurality of unmasked elements of medical time series training data.
